(19) United States
(12) Patent Application Publication
(10) Pub. No.: US 2006/0009755 A1
(43) Pub. Date: Jan. 12, 2006

(54) Method and system for ablation of atrial fibrillation and other cardiac arrhythmias

(76) Inventor: Jasbir S. Sra, Pewaukee, WI (US)

Correspondence Address: JANSSON, SHUPE & MUNGER & ANTARAMIAN, LTD, 245 MAIN STREET, RACINE, WI, 53403, US

(21) Appl. No.: 10/932,490

(22) Filed: Sep. 2, 2004

Related U.S. Application Data
(60) Provisional application No. 60/500,112, filed on Sep. 4, 2003.

(51) Int. Cl. A61B 18/18

(52) U.S. Cl. 606/32; 606/41

(57) ABSTRACT

A method is provided for ablation in treatment of heart arrhythmias such as atrial fibrillation that includes positioning a catheter apparatus with multiple electrodes within a cardiac chamber, visualizing the catheter apparatus with an interventional system, navigating the catheter apparatus within the cardiac chamber, and delivering energy to selected electrodes of the catheter apparatus from an external source to ablate heart tissue at select locations. Preferably, the external source is an external patch placed on the patient for the delivery of radio-frequency energy. The electrodes of the catheter apparatus are connected to the patch through a patient interface unit where the interface unit selects the electrodes to which radio-frequency energy is to be delivered. In another aspect of the invention, a system for ablation of heart arrhythmias is provided that has a catheter apparatus with multiple electrodes, an interventional system for visualizing the catheter apparatus within a cardiac chamber, and an external source for delivering energy to selected electrodes of the catheter apparatus within the cardiac chamber to ablate heart tissue. Preferably, the system further includes a digital imaging system for obtaining cardiac image data, an image generation system for generating a 3D model of the cardiac chamber from the cardiac image data, and a workstation for registering the 3D model with the interventional system and for visualizing the catheter apparatus over this registered 3D model upon the interventional system.

### RELATED APPLICATION

This application claims the benefit of U.S. Provisional Application No. 60/600,112 filed on Sep. 4, 2003.

### FIELD OF THE INVENTION

This invention relates generally to methods and systems for ablation of atrial fibrillation and other cardiac arrhythmias and, in particular, to methods and systems for delivering energy from an outside source to electrodes positioned inside the heart.

### BACKGROUND OF THE INVENTION

Successful ablation of the pulmonary veins, various trigger sites for atrial fibrillation, and other strategic areas within the left atrium through use of a catheter has limitations due to the complex 3D geometry of this heart chamber. One of these limitations involves moving the ablation catheter from one spot to the next within a cardiac chamber. Another difficulty is that inherent limitations of technology, size and geometry prevent multiple electrodes on the catheter from being used to delivery radio-frequency current, either simultaneously or sequentially. Design limitations also contribute to the problem of delivering energy to these different electrodes when positioned inside the heart. There is, therefore, a need for a more innovative delivery process for ablating AF and other heart rhythm problems.

### SUMMARY OF THE INVENTION

One aspect of this invention provides a method for treating a heart arrhythmia in a patient with ablation that includes the steps of (1) positioning a catheter apparatus with multiple electrodes within a chamber of the heart, (2) visualizing the catheter apparatus upon an interventional system such as a fluoroscopic system, (3) navigating the catheter apparatus within this cardiac chamber, and (4) delivering energy to selected electrodes of the catheter apparatus from an external source whereby the electrodes can ablate heart tissue at select locations within the cardiac chamber.

In certain preferred embodiments, the energy delivered by the external source is radio-frequency energy in a manner where the electrodes are inductively coupled to the external source. More preferred is where the external source comprises an external patch placed on the patient, the patch being connected to the electrodes through a patient interface unit. The interface unit can selectively choose the electrodes to which the radio-frequency energy is delivered.

Another desirable embodiment is where the method includes the steps of obtaining cardiac image data from a digital imaging system, generating a 3D model of the cardiac chamber and surrounding structures from this image data, registering the 3D model with the interventional system, visualizing the catheter apparatus over the registered 3D model upon the interventional system, and navigating the catheter apparatus within the cardiac chamber utilizing the registered 3D model.

In a most desirable embodiment, the digital imaging system is a computer tomography (CT) system. Highly desirable is where the heart arrhythmia being treated is atrial fibrillation and the 3D model provides 3D imaging of the left atrium and pulmonary veins.

In another aspect of this invention, a system is provided for treatment of a heart arrhythmia in a patient that has a catheter apparatus with multiple electrodes, an interventional system for visualizing the catheter apparatus within a chamber of the heart, and an external source that delivers energy to select electrodes of the catheter apparatus while inside the cardiac chamber to enable these electrodes to ablate heart tissue at certain chosen locations.

Preferred embodiments find the energy being delivered is radio-frequency energy such that the electrodes are inductively coupled to the external source to receive delivery of this energy. More preferred is where the system has an external patch placed on the patient as the external source and the patch is connected to the electrodes through a patient interface unit. The interface unit permits the electrodes to be selected that are to receive the radio-frequency energy delivered.

Certain desirable embodiments of this system also include a digital imaging system for obtaining cardiac image data, an image generation system for generating a 3D model of the cardiac chamber and surrounding structures from this image data, and a workstation for registering the 3D model with the interventional system and for visualizing the catheter apparatus over the registered 3D model with the interventional system. Most desirable is where the heart arrhythmia is atrial fibrillation and wherein the 3D model is of the left atrium and pulmonary veins. Highly desirable in such systems is where the digital imaging system is a computer tomography (CT) system and the interventional system is a fluoroscopic system.

### BRIEF DESCRIPTION OF THE DRAWINGS

FIG. 1 is a schematic overview of a system for ablation in treatment of a heart arrhythmia in accordance with this invention.

FIG. 2A depicts 3D cardiac images of the left atrium.

FIG. 2B illustrates localization of a standard mapping and ablation catheter over an endocardial view of the left atrium registered upon an interventional system.

FIG. 3 is an illustration of a catheter sheath and catheter with electrodes as it conforms to the 3D geometry of the left atrium.

FIG. 4 is a flow diagram of a method for ablation of atrial fibrillation and other cardiac arrhythmias in accordance with this invention.

### DETAILED DESCRIPTION OF PREFERRED EMBODIMENTS

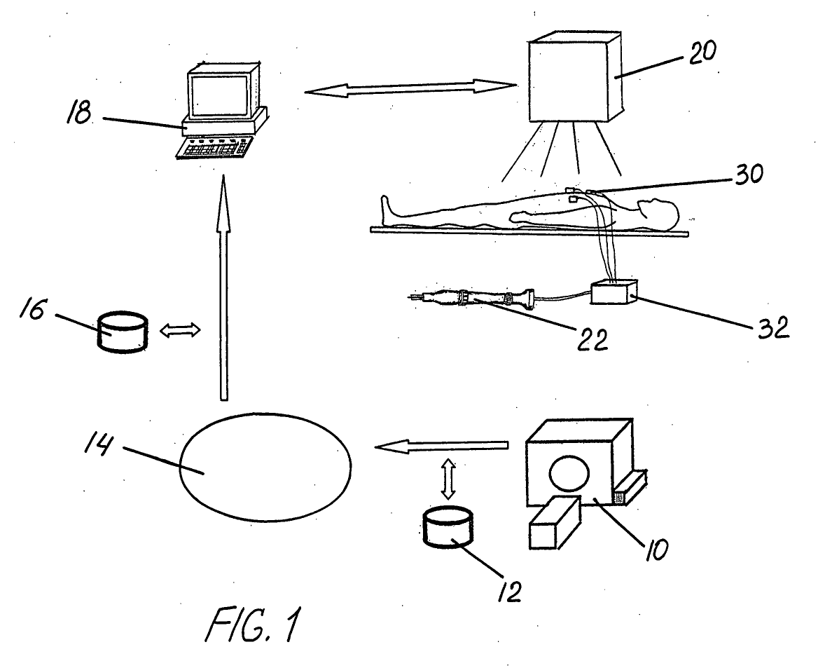

FIG. 1 illustrates a schematic overview of an exemplary system for the ablation of heart tissue in a patient with a heart arrhythmia such as atrial fibrillation in accordance with this invention. A digital imaging system such as a CT scanning system 10 is used to acquire image data of the heart. Although the embodiments discussed hereinafter are described in the context of a CT scanning system, it will be appreciated that other imaging systems known in the art, such as MRI and ultrasound, are also contemplated.

Cardiac image data 12 is a volume of consecutive images of the heart collected by CT scanning system 10 in a continuous sequence over a short acquisition time. The shorter scanning time through use of a faster CT scanning system and synchronization of the CT scanner with the QRS on the patient's ECG signal reduces the motion artifacts in images of a beating organ like the heart. The resulting cardiac image data 12 allows for reconstruction of images of the heart that are true geometric depictions of its structures.

Cardiac image data 12 is then segmented using protocols optimized for the left atrium and pulmonary arteries by image generation system 14. It will be appreciated that other chambers of the heart and their surrounding structures can be acquired in a similar manner. Image generation system 14 further processes the segmented data to create a 3D model 16 of the left atrium and pulmonary arteries using 3D surface and/or volume rendering. Additional post-processing can be performed to create navigator (view from inside) views of these structures.

3D model 16 is then exported to workstation 18 for registration with an interventional system such as a fluoroscopic system 20. The transfer of 3D model 16, including navigator views, can occur in several formats such as the DICOM format and geometric wire mesh model. Information from CT scanning system 10 will thus be integrated with fluoroscopic system 20. Once 3D model 16 is registered with fluoroscopic system 20, 3D model 16 and any navigator views can be seen on the fluoroscopic system 20.

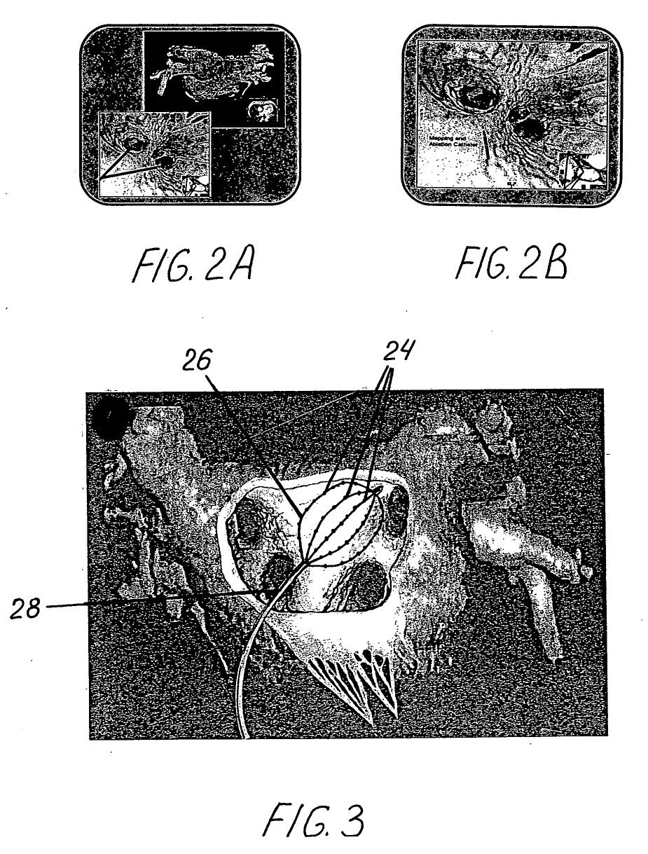

A detailed 3D model of the left atrium and the pulmonary veins, including endocardial or inside views, is seen in FIG. 2A. The distance and orientation of the pulmonary veins and other strategic areas can be calculated in advance from this 3D image to create a roadmap for use during the ablation procedure.

Using a transeptal catheterization, which is a standard technique for gaining access to the left atrium, a catheter apparatus 22, having a mapping and ablation catheter 26 with multiple electrodes 24, is introduced into the left atrium. Catheter 26 is visualized on the fluoroscopic system 20 over the registered 3D model 16. Catheter 26 is then navigated real time over 3D model 16 to the appropriate site within the left atrium. FIG. 2B illustrates localization of a standard mapping and ablation catheter over an endocardial view of the left atrium registered upon an interventional system.

Electrodes 24 of catheter apparatus 22 are capable of both mapping and ablation. Electrodes 24 are spaced apart along catheter 26 of the catheter apparatus 22 and are fabricated from commercially available conductive material such as platinum or copper. Preferably, each electrode 24 will be about 2 mm in size but it will be appreciated that different shapes and sizes can be used as needed. The electrodes are positioned upon a spline made from commercially available material such as stainless steel or nitinol.

Catheter 26 has at least 60 electrodes 24 capable of delivering energy; however, more can be used as needed. Catheter sheath 28 of catheter apparatus 22 encloses catheter 26 until sheath 28 has been placed inside the left atrium or other heart chamber of interest. Inside the left atrium, catheter 26 is projected outward from sheath 28. Catheter 26 expands upon exiting sheath 28 to conform to the 3D anatomy of the left atrium.

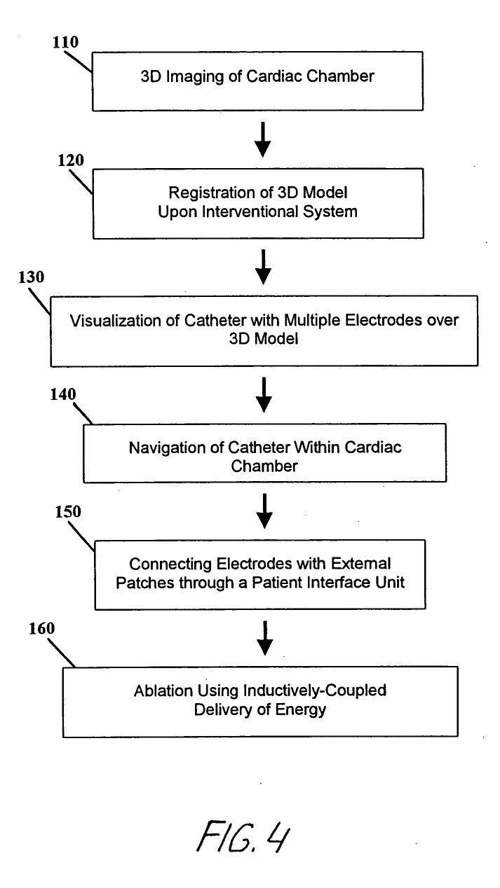

FIG. 3 illustrates, as an example, the introduction of catheter 26 into the left atrium using the transeptal approach and shows how catheter 26 expands in conformity to the 3D left atrial anatomy. FIG. 3 presents the anterior view of the left atrium with the right pulmonary veins on the left side and left pulmonary veins on the right side. As illustrated, catheter sheath 28 can be adjusted to achieve different orientations before catheter 26 is deployed depending upon the pulmonary veins or other strategic areas that need to be accessed. Once catheter sheath 28 has been placed in the desired orientation, catheter 26 can be extended outward.

The structure and configuration of catheter 26 can vary to accommodate different atrial or other chamber sizes. Such structures include one where catheter 26 expands inside the left atrium into the shape of a basket as shown in FIG. 3 with multiple electrodes 24 secured along its length.

One or more external patches 30 are then positioned on the surface of the body of the patient as illustrated in FIG. 1. Patches 30 are connected to electrodes 24 of catheter apparatus 22 through a patient interface unit 32. Patient interface unit 32 is electrically linked to an external generator (not shown). Patches 30 direct radio-frequency energy to certain selected electrodes 24 inside the heart using inductively coupled delivery of the radio-frequency current.

Intracardial recordings and real-time visualizations of catheter 26 over the registered 3D model with the fluoroscopic system 20 permit a determination of which electrodes 24 are to be used for ablation. The externally controlled circuitry of patient interface unit 32 is programed with a map of electrodes 24 to enable unit 32 to identify the precise electrodes 24 to which radio-frequency energy needs to be delivered. One or more electrodes 24 can be used simultaneously for ablation. Patient interface unit 32 can be operated manually by the physician or provided with predetermined programs that the physician can select from to modify or operate automatically.

One skilled in the art will recognize that delivery of radio-frequency energy utilizing external patches 30 can also be accomplished when the catheter apparatus 22 is visualized and navigated within a cardiac chamber using an interventional system such as fluoroscopy but without any registered 3D models or images.

There is shown in FIG. 4 an overview of a method for ablation of atrial fibrillation and other cardiac arrhythmias in accordance with this invention. As seen in step 110, a 3D image of the heart is obtained from which a 3D model of the chamber of interest is created through segmentation of the image data using protocols optimized for the appropriate structures. 3D images of the heart can be acquired using CT scan or MRI. Once this 3D model has been obtained, it can be stored as an electronic data file using various means of storage. The stored model can then later be transferred to a computer workstation linked to an interventional system.

As illustrated in step 120, after it has been transferred to the workstation, the 3D model is registered with the interventional system. The registration process allows medical personnel to correlate the stored 3D image of the cardiac chamber with the interventional system which is being used with a particular patient. The process also allows the physician to select a catheter that is the proper configuration for the cardiac chamber being ablated. This permits the portion of the catheter apparatus having electrodes to be tailored for the specific arrhythmia and for the specific anatomy of that chamber of the heart.

The next step 130 involves visualization of the catheter over the 3D model registered upon the interventional system. Thus at step 140, as the catheter is navigated inside the chamber, the position and location of the electrodes is superimposed on the 3D image such that medical personnel can accurately localize the electrode or electrodes for ablation at the desired location.

In step 150, external patches are placed on the patient. These patches are connected to the multiple electrodes of the mapping and ablation catheter inside the cardiac chamber of interest through a patient interface unit. The patient interface unit is configured in such a way that its external circuitry can be used to direct radio-frequency energy to the desired electrodes inside the heart.

As seen in step 160, ablation of heart tissue at specifically selected locations is accomplished using ablation electrodes that receive their energy through the inductively coupled delivery of radio-frequency current. The use of external patches and the inductive coupled delivery of radio-frequency energy allows the catheter apparatus to perform additional functions, especially ones that utilize the 3D model registered upon the interventional system.

Various alternatives and embodiments are contemplated as being within the scope of the following claims particularly pointing out and distinctly claiming the subject matter regarded as the invention.

1. A method for ablation in treatment of a heart arrhythmia in a patient comprising:
positioning a catheter apparatus with multiple electrodes within a cardiac chamber;
visualizing the catheter apparatus with an interventional system;
navigating the catheter apparatus within the cardiac chamber; and
delivering energy to selected electrodes of the catheter apparatus from an external source to ablate heart tissue at select locations.

2. The method of claim 1 wherein the energy delivered is radio-frequency energy, whereby the electrodes are inductively coupled to the external source.

3. The method of claim 2 wherein the external source is an external patch placed on the patient, the patch being connected to the electrodes through a patient interface unit, whereby the interface unit selects the electrodes to which radio-frequency energy is delivered.

4. The method of claim 3 wherein the interventional system is a fluoroscopic system.

5. The method of claim 1 further comprising the steps of:
obtaining cardiac image data from a digital imaging system;
generating a 3D model of the cardiac chamber and surrounding structures from the cardiac image data;
registering the 3D model with the interventional system;
visualizing the catheter apparatus over the registered 3D model with the interventional system; and
navigating the catheter apparatus within the cardiac chamber utilizing the registered 3D model.

6. The method of claim 5 wherein the digital imaging system is a computer tomography (CT) system.

7. The method of claim 6 wherein the heart arrhythmia is atrial fibrillation and wherein the 3D model is of the left atrium and pulmonary veins.

8. The method of claim 7 wherein the energy delivered is radio-frequency energy, whereby the electrodes are inductively coupled to the external source.

9. The method of claim 8 wherein the external source is an external patch placed on the patient, the patch being connected to the electrodes through a patient interface unit, whereby the interface unit selects the electrodes to which radio-frequency energy is delivered.

10. The method of claim 9 wherein the interventional system is a fluoroscopic system.

11. A system for ablation in treatment of a heart arrhythmia in a patient comprising:
a catheter apparatus having multiple electrodes;
an interventional system for visualizing the catheter apparatus within a cardiac chamber; and
an external source for delivering energy to selected electrodes of the catheter apparatus within the cardiac chamber to ablate heart tissue at select locations.

12. The system of claim 11 wherein the energy delivered is radio-frequency energy, whereby the electrodes are inductively coupled to the external source.

13. The system of claim 12 wherein the external source is an external patch placed on the patient, the patch being connected to the electrodes through a patient interface unit, whereby the interface unit selects the electrodes to which radio-frequency energy is delivered.

14. The system of claim 13 wherein the interventional system is a fluoroscopic system.

15. The system of claim 11 further comprising:
a digital imaging system for obtaining cardiac image data;
an image generation system for generating a 3D model of the cardiac chamber and surrounding structures from the cardiac image data; and
a workstation for registering the 3D model with the interventional system and for visualizing the catheter apparatus over the registered 3D model with the interventional system.

16. The system of claim 15 wherein the digital imaging system is a computer tomography (CT) system.

17. The system of claim 16 wherein the heart arrhythmia is atrial fibrillation and wherein the 3D model is of the left atrium and pulmonary veins.

18. The system of claim 17 wherein the energy delivered is radio-frequency energy, whereby the electrodes are inductively coupled to the external source.

19. The system of claim 18 wherein the external source is an external patch placed on the patient, the patch being connected to the electrodes through a patient interface unit, whereby the interface unit selects the electrodes to which radio-frequency energy is delivered.

20. The system of claim 19 wherein the interventional system is a fluoroscopic system.
